(19) United States
(12) Patent Application Publication
(10) Pub. No.: US 2012/0179066 A1
(43) Pub. Date: Jul. 12, 2012

(54) SLEEPING QUALITY MONITOR SYSTEM AND A METHOD FOR MONITORING A PHYSIOLOGICAL SIGNAL

(75) Inventors: YEH-LIANG HSU, TAOYUAN COUNTY (TW); CHANG-HUEI WU, TAOYUAN COUNTY (TW); CHIH-MING CHENG, TAOYUAN COUNTY (TW); HONG-XIANG MA, TAOYUAN COUNTY (TW); ZHI-WEI RUAN, TAOYUAN COUNTY (TW)

(73) Assignee: YUAN ZE UNIVERSITY, TAOYUAN COUNTY (TW)

(21) Appl. No.: 13/424,606

(22) Filed: Mar. 20, 2012

Related U.S. Application Data
(63) Continuation-in-part of application No. 12/289,858, filed on Nov. 6, 2008.
Application No. 12/289,858 is a division of application No. 11/582,422, filed on Oct. 18, 2006, now abandoned.

(51) Int. Cl. A61B 5/00; A61B 7/00; A61B 5/11

(52) U.S. Cl. 600/586; 600/300; 600/595

(57) ABSTRACT

A method for monitoring a physiological signal monitors the sleeping quality of a person under sleep test with long hours in daily life at home. The system comprises a distributed data server, at least one physiological signal sensor, and at least one computer. The sensor is wired or wirelessly connected to the server. The server connects on Internet to the computer. The sensor transmits the sensed physiological signals to the server to process, calculate, analyze, and store. By means of physiological signal data processing, the computer further calculates and analyzes the physiological signal data stored in the server and allows an authorized reader to read the result. With the household sensor working with the server connecting to Internet, the system may be available in daily life at home to monitor the sleeping quality of the person under sleep test with long hours.

### CROSS-REFERENCE TO RELATED APPLICATION

This U.S. Non-provisional Application for Patent is a continuation-in-part application of patent application Ser. No. 12/289,858 filed on 6 Nov. 2008, currently pending, which is a Divisional patent application of Ser. No. 11/582,422, filed on 18 Oct. 2006, which is abandoned. The entirety of each of the above-mentioned patent applications is hereby incorporated by reference herein and made as a part of this specification.

### BACKGROUND OF THE INVENTION

1. Field of the Invention

This invention relates to a sleeping quality monitor system and a method for monitoring a physiological signal, and particularly to a sleeping quality monitor system applicable in daily life at home and suitable for monitoring the sleeping quality of a person under sleep test with long hours and a method for monitoring a physiological signal thereof.

2. Description of Related Art

Generally, people spend one-third time in sleeping every day, but unhealthy sleep or insomnia is heavily harmful to them on physiology and psychology. Many people are troubled with sleep; even if they may easily fall asleep, the trouble of physiological reaction during sleep will also affect their physiological and physiological conditions and even threaten the lives, such as snoring. People who snore during sleep sometimes also stop breathing. Even if having enough sleeping time, yet due to awakening caused by many times of asphyxiation during sleep, people still feel insufficient sleep, which may seriously affect their brains and hearts on account of long-term anoxia.

A conventional sleeping quality monitor system uses a set of overnight polysomnography to monitor the times and types of breathe pause and shallow breath in a period of overnight sleep, the indices and times of anoxia, the variation of electrocardiogram, the airflow of mouth cavity and nasal cavity, the breathe running of chest and belly, oxygen content in blood, the times of snore as physiological conditions, and even extra instruments may be added to monitor the physiological items of the person under sleep test based on his or her conditions. The system is sky-high precise and featured with wide monitor, so results given by the system are very accurate and close-knit, which is quite helpful to diagnosis on a disease. However, the system must be executed only in a medical institution or at a laboratory of an academic unit, so it is not suitable for long-term and universal monitor. Thus, only when being under the weather or infected with a disease, people in general leave for the medical institution for health check.

For this reason, in consideration of improving the defects described above, the inventor, having concentrated their studies and operating in coordination with academic theories, has finally provided this invention as a reasonable design and an effective improvement over the defects mentioned above.

### SUMMARY OF THE INVENTION

This invention is mainly to provide a sleeping quality monitor system and a method for monitoring a physiological signal, which may be used to monitor the sleeping quality of a person under sleep test with long hours in daily life at home, detect the disturbance of sleeping of the person under sleep test at an early date for speedy improvement, and thus prevent a disease caused by the disturbance of sleeping and the physiological abnormality during sleep.

In order to achieve the object, in this invention, a sleeping quality monitor system is provided, comprising a distributed data server, at least one physiological signal sensor, and at least one computer. The physiological signal sensor is wired or wirelessly connected to the distributed data server and transmits monitored physiological signals to the distributed data server to process, calculate, analyze, and finally store. The computer connects on Internet to the distributed data server, physiological signal data stored in the server may be read through the computer on Internet, and then by means of physiological signal data processing, the physiological signal data is calculated and analyzed and then displayed on a PC screen.

In this invention, a method for monitoring a physiological signal at home is provided. The method being used to monitor physiological parameters of a person under sleep test for determination of his or her sleeping quality by executed in a distributed data server and an electronic device, the steps comprising: A) using a sensor to sense the person under sleep test, and then obtaining a plurality of physiological signals; B) receiving the sensed physiological signals by a signal processing module of the distributed data server; C) processing the sensed physiological signals by the signal processing module; D) analyzing the processed physiological signals to generate a plurality of statistical values according to a threshold limit value by the signal processing module; E) calculating the statistical values via at least one formula to generate a plurality of weighted values by the signal processing module; F) comparing the weighted values and a sleeping state index to determine a plurality of sleeping quality information by the signal processing module; G) storing the sleeping quality information by a storage module of the distributed data server; and H) reading the sleeping quality information by the electronic device.

In order to further know the features and technical means of this invention, refer to the detailed description according to this invention accompanied with drawings; however, the accompanied drawings are provided for reference and illustration only and are not limited to this invention.

### BRIEF DESCRIPTION OF THE DRAWINGS

FIG. 1 is a schematic view illustrating a system structure of an embodiment of this invention;

FIG. 1A is a functional block diagram illustrating the system structure of the embodiment of this invention;

FIG. 1B a functional block diagram illustrating the system structure of the embodiment of this invention when the sensor is used for sensing the body movement of the person;

FIG. 1C a functional block diagram illustrating the system structure of the embodiment of this invention when the sensor is used for receiving the snore of the person;

FIG. 2 is a flow chart of descriptive blocks of the system of the embodiment of this invention;

FIG. 3 is a schematic view illustrating the system structure of the other embodiment of this invention; and

FIG. 4 is a flow chart of descriptive blocks of the system of the other embodiment of this invention.

### DETAILED DESCRIPTION OF THE PREFERRED EMBODIMENTS

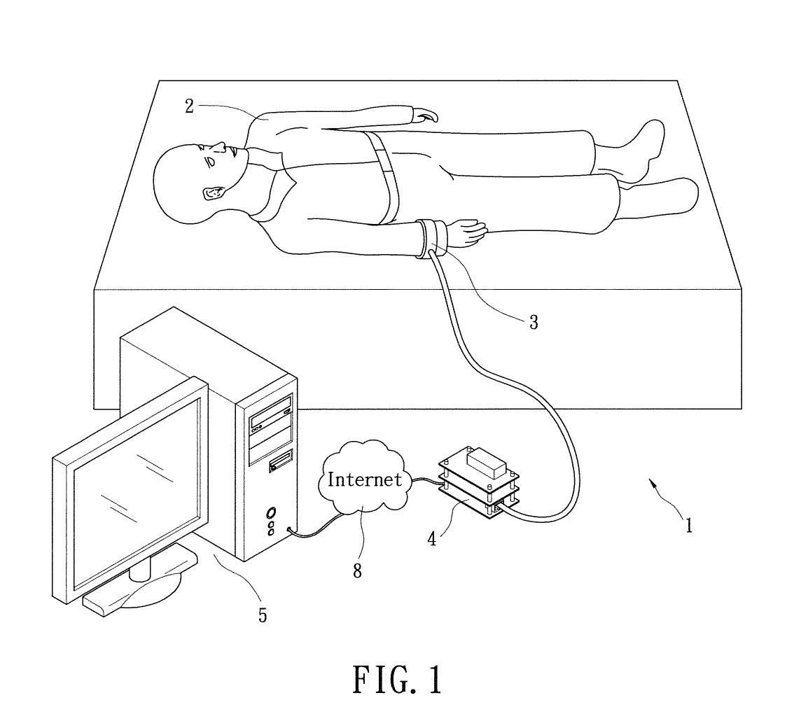

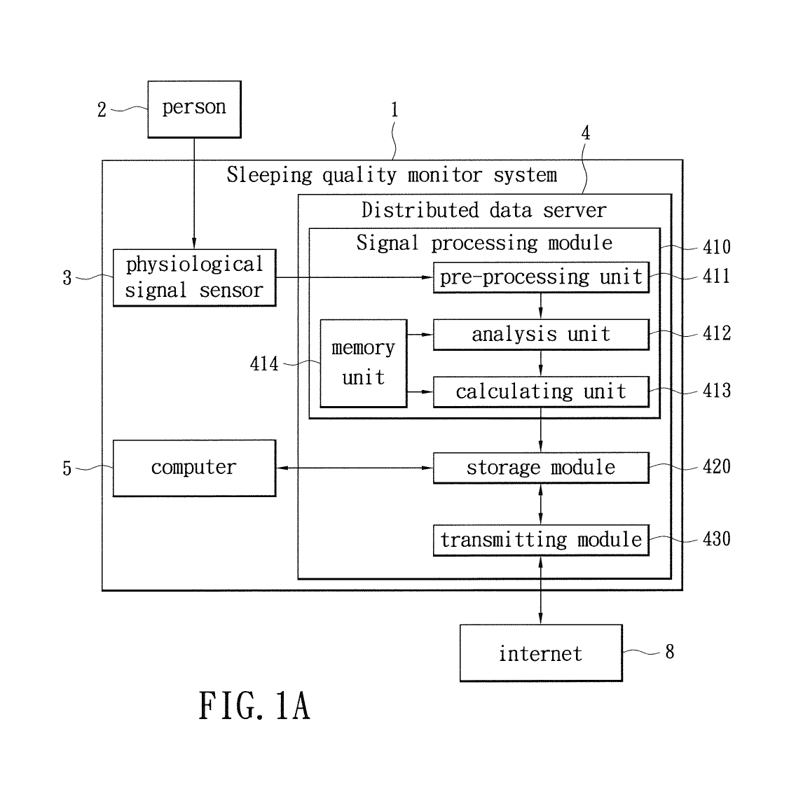

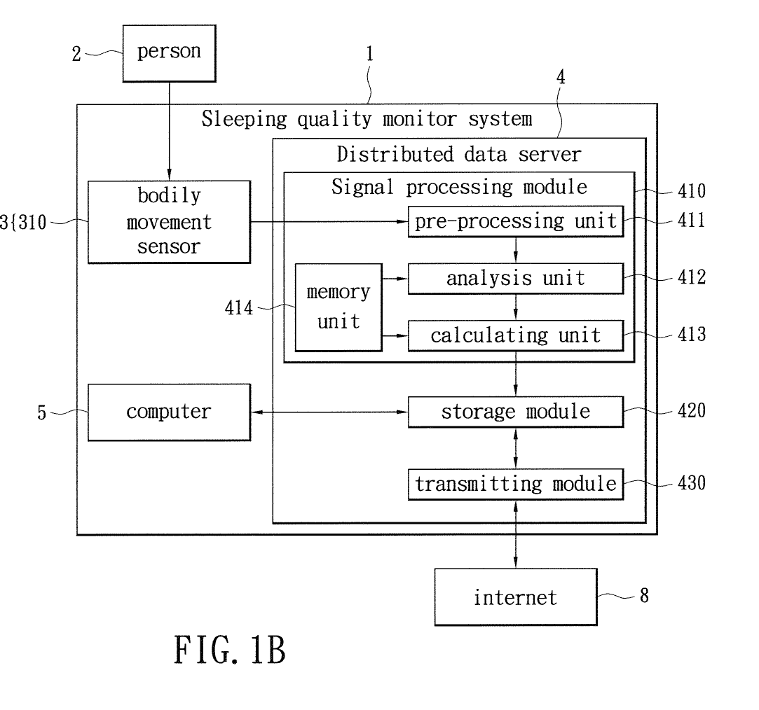

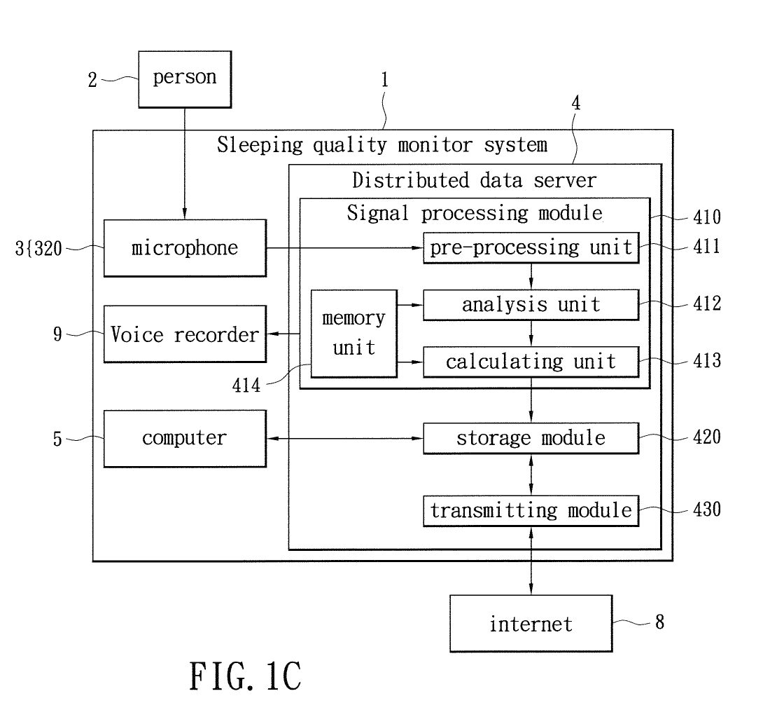

With reference to FIG. 1, a sleeping quality monitor system is provided in this invention to monitor the physiological signal of a person under sleep test for determining his or her sleeping quality. The sleeping quality monitor system 1 used for disposed at the person's home, comprises at least one physiological signal sensor 3, a distributed data server 4 (DDS), and at least one computer 5.

The physiological signal sensor 3 may be wired through a transmission line or wirelessly connected through RS-232 as a wireless transmission module to the distributed data server 4. The computer 5 is wired or wirelessly connected to Internet 8 and then connected to the distributed data server 4 on Internet. The physiological signal sensor 3 senses physiological signals, such as snore, breathe cycles per second, body movement or temperature and the like, and may be called a stertor sensor, a breathe cycle sensor, a body movement sensor, or a body temperature sensor, and they are household physiological signal sensor.

On the other hand, the distributed data server 4 initially process, calculate, and analyze the physiological signals and then store them therein. And the data analyzed is also used to determine the physiological conditions of person under sleep test. If finding abnormality, the distributed data server 4 will send an alarm message to a receiving end configured in advance. Further, the person under sleep test may also send a call message actively, and when receiving the message, the server 4 will also send an alarm message to the receiving end 6. The relatives of the person under sleep test, the person under sleep test, or his or her doctor as an authorized agent may read the physiological signal data stored in the server 4 through the physiological signal data processed by the computer 5, and after being further calculated and analyzed, the data of person under sleep test may be read.

Please refer to FIG. 1A, which shows the functional diagram about the sleeping quality monitor system 1. Specifically, the DDS 4 has a signal processing module 410, a storage module 420, and a transmitting module 430, wherein the signal processing module 410 has a pre-processing unit 411, an analysis unit 412, a calculating unit 413, and a memory unit 414.

The pre-processing unit 411 is electrically connected to the physiological signal sensor 3. The analysis unit 412 is electrically connected between the pre-processing unit 411 and the calculating unit 413. The storage module 420 is electrically connected between the calculating unit 413 and the transmitting module 430. Moreover, the memory unit 414 is electrically connected to the analysis unit 412 and the calculating unit 413, and the memory unit 414 has a threshold limit value (TLV), at least one formula, and at least one sleeping state index.

The physiological signal sensor 3 transmits the physiological signals 21 to the signal processing module 410. The pre-processing unit 411 converts the physiological signals 21 into digital formation, and then the physiological signals 21 is filtered to cause the waveform of the physiological signals 21 smoother.

The analysis unit 412 receives the physiological signals 21, which after processing from the pre-processing unit 411, and analyzes the variation of each physiological signal 21 as time goes on. The analysis unit 412 classifies and counts the variations of the physiological signals 21 according to the TLV of the memory unit 414 to generate a plurality of statistical values.

The calculating unit 413 receives the statistical values and calculates the statistical values by the formula of the memory unit 414 to generate a plurality of weighted values. The calculating unit 413 compares the weighted values and the sleeping state index of the memory unit 414 to determine a plurality of sleeping quality information. The storage module 420 receives and stores the sleeping quality information.

The relatives of the person under sleep test, the person under sleep test, or his or her doctor as an authorized agent may read the sleeping quality information stored in the storage module 420 of the server 4 via the transmitting module 430 or the computer 5.

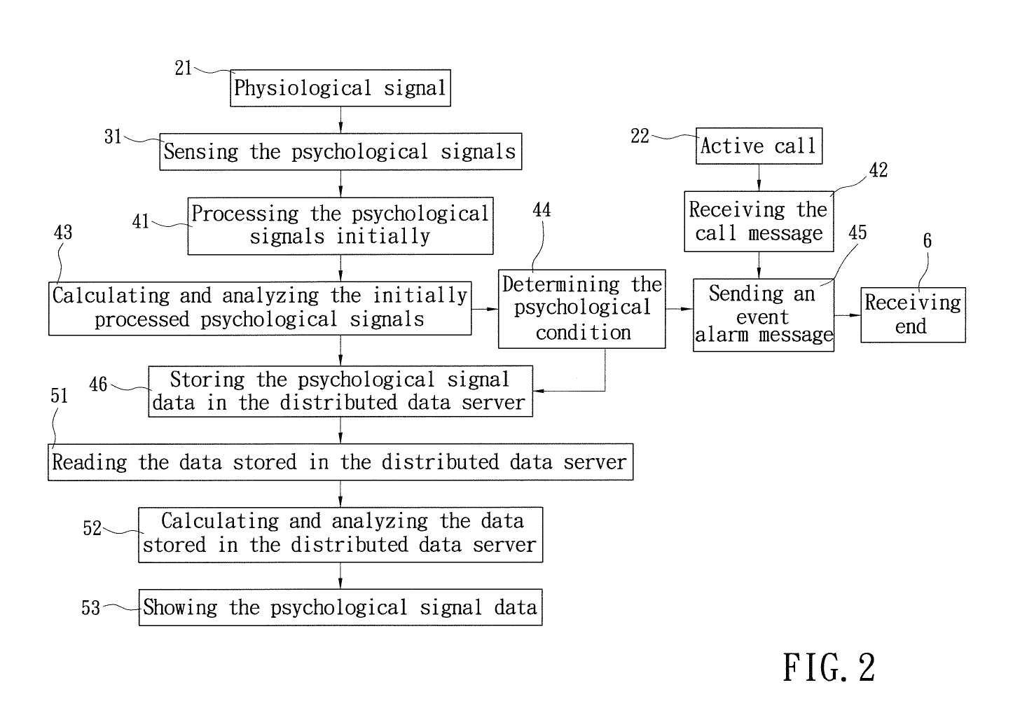

With reference to FIGS. 1 and 2, a method for monitoring the physiological signal is further provided in this invention to monitor the physiological signals 21 of the person under sleep test 2 for determination of his or her sleeping quality. At first, the physiological signal sensor 3 senses the physiological signals 31. At the step, the physiological signal sensor 3 senses the physiological signals 21 of the person under sleep test 2 and then transmits the physiological signals 21 to the distributed data server 4 to process physiological signals initially 41. Next, likewise in the distributed data server 4, the transmitted physiological signals 21 are calculated and analyzed 43. At the step, the distributed data server 4 in advance briefly calculates and analyzes the processed physiological signals 21 to get a physiological signal data, the same as the sleeping quality information. Next, the physiological signal data is stored in the distributed data server 46, and the data stored at this time is the original point data of the physiological signals and data having the physiological meaning. Then, the authorized reader reads the data stored in the distributed data server 51 by means of the physiological signal data processing in the computer 5, and reads the physiological signal data stored in the distributed data server 4 on Internet 8. The means of the physiological signal data processing calculates and analyzes the data stored in the distributed data server 52 so that the reader may read what he or she want to in a chart. Finally, at the step of showing the physiological signal data 53, the chart of data is shown on a PC screen on which the authorized reader may read.

Further, after the step of calculating and analyzing the initially processed physiological signals 43, the distributed data server 4 stores the physiological signal data and meanwhile determines the result given from the pieces of data. At the step of determining the physiological condition 44, if it is found that the physiological signal of the person under sleep test 2 is a condition in advance set up for an alarm, such as a higher temperature or physiological conditions of the person under sleep test 2 that are same with long hours and regarded as an abnormal condition, such as no movement but breathe; in these two conditions, the distributed data server 4 sends an alarm message 45 to a receiving end 6 set up in advance. The alarm message may be sent to a mobile phone or a PDA working with a cell phone number or to an e-mail box. Besides, the person under sleep test 2 may also send a call message in the manner of active call 22. From the distributed data server 4, the call message is received 42 and then sent to the receiving end 6.

More detail, please refer to FIG. 1B, which shows that the physiological signal sensor 3 takes a bodily movement sensor 310 for example. The bodily movement sensor 310 is used for sensing the movement about arm, body, or leg of the person 2, or sensing the respiratory rate or body temperature of the person 2.

The bodily movement sensor 310 is used for sensing the body movement of the person 2 to obtain a plurality of physiological signals 21. The bodily movement sensor 310 transmits the physiological signals 21 to the signal processing module 410. The pre-processing unit 411 converts the physiological signals 21 into digital formation, and then the physiological signals 21 is filtered to cause the waveform of the physiological signals 21 smoother.

The analysis unit 412 receives the physiological signals 21, which after processing from the pre-processing unit 411, and analyzes the slope-variation of each physiological signal 21 as time goes on. The analysis unit 412 classifies the slope-variations of the physiological signals 21 according to the TLV (such as 0.02) of the memory unit 414, and counts the time of the slope-variations, which over the TLV in every one minute, to generate a plurality of first statistical values (Bᵢ). In other words, the first statistical value (Bᵢ) is presented the moving time of the body of the person 2 in the ‘i th’ minute.

The calculating unit 413 receives the first statistical values (Bᵢ) and calculates the first statistical values (Bᵢ) by a weighted formula of the memory unit 414 to generate a plurality of first weighted values (wBᵢ). The weighted formula is as follows:

$${wB}_{i} = ( k - n + 1 ) {\sum }_{n = 1}^{k} {B}_{i - n + 1} / {\sum }_{n = 1}^{k} n$$

In the weighted formula, ‘k’ is total time of the sleep test, and ‘Bᵢ’ means the time of the variations, which over the threshold limit value, in the ‘i th’ minute counting from the start of the sleep test. Likewise, the signal processing module 410 can get a plurality of physiological signals 21 about leg movement of the person 2, an then obtain a plurality of second statistical values (Lᵢ) and second weighted values (wLᵢ).

The second weighted values (wLᵢ) is obtained from a weighted formula as follows:

$${wL}_{i} = ( k - n + 1 ) {\sum }_{n = 1}^{k} {L}_{i - n + 1} / {\sum }_{n = 1}^{k} n$$

The calculating unit 413 calculates the first weighted values (wBᵢ) and the second weighted values (wLᵢ) by a sleep-depth formula of the memory unit 414 to get a plurality of SleepDepth values corresponding to every one minute. The sleep-depth formula is as follows:

SleepDepth value=0.659−0.028wBᵢ−0.026wLᵢ

The calculating unit 413 compares the SleepDepth values and the sleeping state index of the memory unit 414 to determine a plurality of sleeping quality information.

In addition, please refer to FIG. 1C, which shows that the physiological signal sensor 3 takes a microphone 320 for example. The microphone 320 is used for receiving the snore as the person 2 sleeping.

The microphone 320 is used for receiving the snore of the person 2 to obtain a plurality of physiological signals 21. The microphone 320 transmits the physiological signals 21 to the signal processing module 410. The pre-processing unit 411 converts the physiological signals 21 into digital formation, and then the physiological signals 21 is filtered to cause the waveform of the physiological signals 21 smoother.

The analysis unit 412 receives the physiological signals 21, which after processing from the pre-processing unit 411, and analyzes the slope-variation of each physiological signal 21 as time goes on. The analysis unit 412 classifies the slope-variations of the physiological signals 21 according to the TLV of the memory unit 414, and counts the number of the slope-variations, which over the TLV in every one minute, to generate a plurality of third statistical values (Sᵢ). In other words, each third statistical value (Sᵢ) is presented the snoring frequency of the person 2 at every one minute.

In addition, the analysis unit 412 further classifies the third statistical values (Sᵢ) to a general snoring and an intermittent snoring, wherein the interval time between two snores over 10 seconds is defined the intermittent snoring. And then, the analysis unit 412 counts the numbers of the intermittent snoring.

The calculating unit 413 receives the third statistical values (Sᵢ), and calculates the numbers of the intermittent snoring to obtain a plurality of intermittent snoring ratios (ISᵢ), which is presented the intermittent snoring frequency of the person 2 in every one minute.

The calculating unit 413 calculates the intermittent snoring ratios (ISᵢ) by a weighted formula of the memory unit 414 to generate a plurality of third weighted values (wBᵢ). The weighted formula is as follows:

$${wIS}_{i} = ( k - n + 1 ) {\sum }_{n = 1}^{k} {IS}_{i - n + 1} / {\sum }_{n = 1}^{k} n$$

The weighted formula taking five minutes for example (k=5) is as follows:

$${wIS}_{i} = \frac{5}{15} {IS}_{i} + \frac{4}{15} {IS}_{i - 1} + \frac{3}{15} {IS}_{i - 2} + \frac{2}{15} {IS}_{i - 3} + \frac{1}{15} {IS}_{i - 4}$$

The calculating unit 413 compares the third weighted values (wISᵢ) and the sleeping state index of the memory unit 414 to determine a plurality of sleeping quality information.

Moreover, the sleeping quality monitor system 1 further has a voice recorder 9 electrically connected to the signal processing module 410. The voice recorder 9 is used for recording the snore of the person 2.

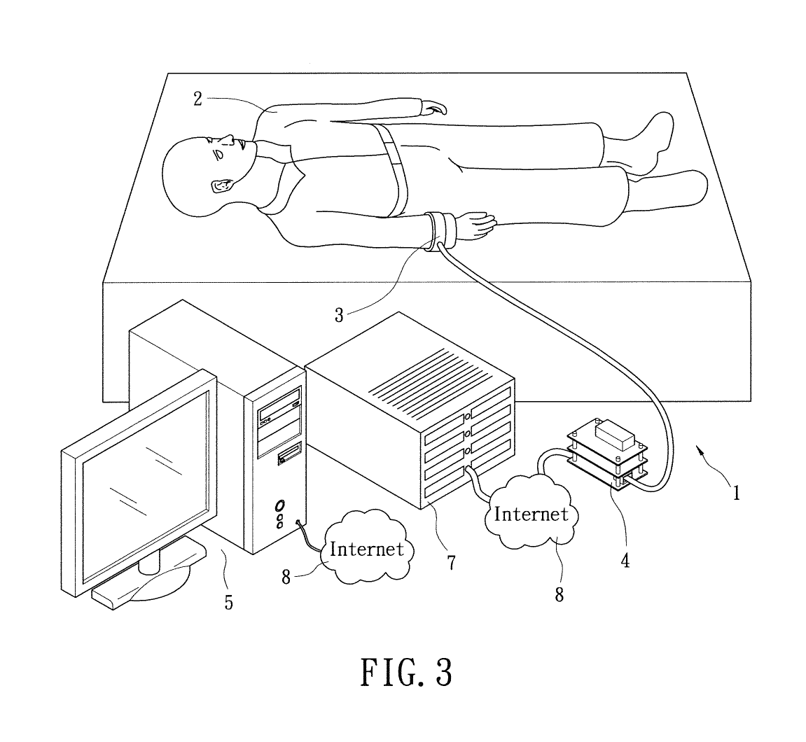

With reference to FIG. 3, in the embodiment, an application server 7 is mainly added to the sleeping quality monitor system 1. The application server 7 is wired or wirelessly connected to Internet 8 and then to the distributed data server 4. The application server 7 may read the physiological signal data stored in the distributed data server 4 by means of the physiological signal data processing and further calculate and analyze it and next stored it, and may manage the distributed data server 4 by means of system management and maintain and update the distributed data server 4. The computer 5 is also connected on Internet to the application server 7. Browsing from the computer 5 on Internet, the relatives of the person under sleep test 2, the person under sleep test 2, or his or her doctor as an authorized agent may read the physiological signal data stored in the application server.

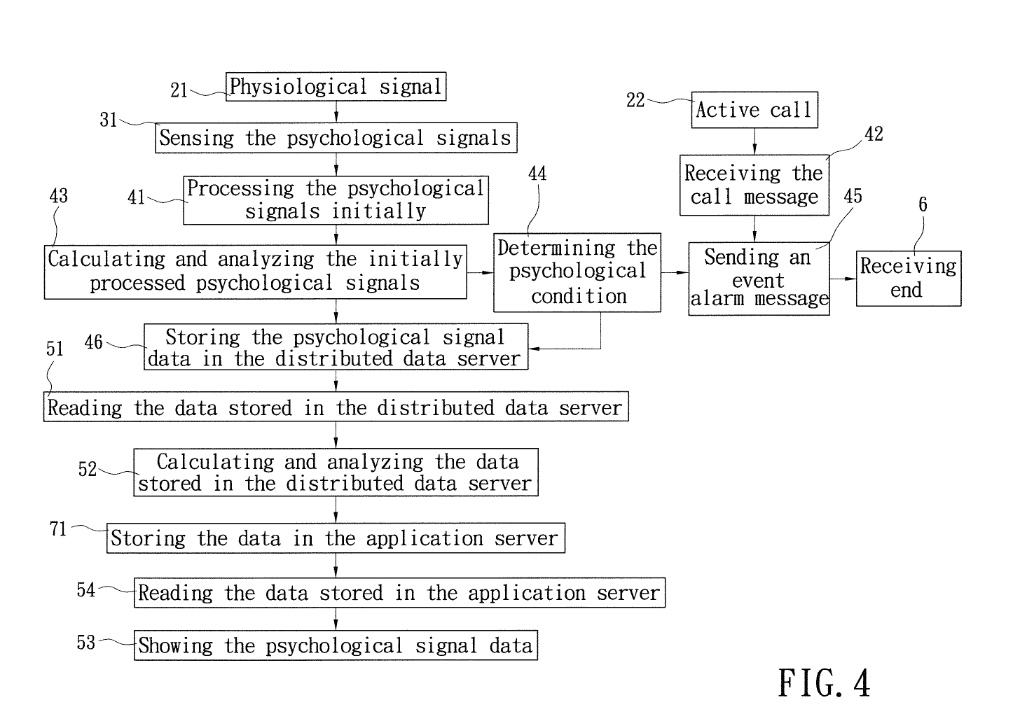

With reference to FIGS. 3 and 4, in the embodiment, a step 71 of storing the data in the application server 7 is added and a step 54 of reading the data stored in the application server from the computer 5. Thus, by means of the physiological signal data processed by the application server 7, the data stored in the distributed data server 4 controls the application server 7 that reads the data 51 stored in the distributed data server 4, calculates and analyzes the data 52 stored in the distributed data server, and stores the data in the application server 71. At the three steps, the step 51 of reading data stored in the distributed data server is to read the physiological signal data stored in the distributed data server 4; also, for calculating and analyzing the data stored in the distributed data server 52 is to calculate and analyze the read physiological signal data stored in the distributed data server 4 for making various forms of charts. The chart is made for the reader to directly read. The data is first stored in the application server 7 and the reader reads the data stored in the application server 54 via the computer 5 on Internet by a mean of Internet navigating and watches the physiological signal data by a mean of showing the physiological signal data 53. The application server 7 is not confined in correspondence to a single distributed data server 4 and may meanwhile read the data stored in many distributed data servers 4 for achievement of management of a large number of data.

Further, in the two embodiments, by means of the physiological signal data processing and Internet navigating, real-time sensed data extracted by the physiological signal sensor 3 may be given so that the relatives or doctor of the person under sleep test 2 may read real-time physiological variation and dispose of the abnormality.

In the embodiments, the physiological signal sensor 3 is used to sense physiological signals, such as snore, breathe cycles per second, body movement or temperature and the like. With the physiological signals, it is determined that the person under sleep test 2 is awake or falls asleep or even deep asleep or shallow asleep at the present time, and thereby lay-up time, incubation period of sleep, sleep efficiency, times of awakening at the mid-night and the like as sleeping quality indices are given for evaluation of the sleeping quality of the person under sleep test 2. With the household physiological signal sensor 3 working with the distributed data server 4 connecting to Internet 8, the sleeping quality may be monitored over a long period of time in daily life at home so that the disturbance of sleeping and the physiological abnormality during sleep may be found and improved early.

However, in the description mentioned above, only the preferred embodiments according to this invention are provided without limit to claims of this invention; all those skilled in the art without exception should include the equivalent changes and modifications as falling within the true scope and spirit of the present invention.

What is claimed is:

1. A method for monitoring a physiological signal at home, said method being used to monitor physiological parameters of a person under sleep test for determination of his or her sleeping quality by executed in a distributed data server and an electronic device, the steps comprising:
A) sensing the person under sleep test through a sensor to obtain a plurality of physiological signals;
B) receiving the sensed physiological signals by a signal processing module of the distributed data server;
C) processing the sensed physiological signals by the signal processing module;
D) analyzing the processed physiological signals to generate a plurality of statistical values according to a threshold limit value by the signal processing module;
E) calculating the statistical values via at least one formula to generate a plurality of weighted values by the signal processing module;
F) comparing the weighted values and a sleeping state index to determine a plurality of sleeping quality information by the signal processing module;
G) storing the sleeping quality information by a storage module of the distributed data server; and
H) reading the sleeping quality information by the electronic device.

2. The method for monitoring a physiological signal according to claim 1, wherein the distributed data server has a pre-processing unit, and in the step C), the pre-processing unit is used for receiving the sensed physiological signals from the sensor, converting the sensed physiological signals into digital formation, and filtering the physiological signals.

3. The method for monitoring a physiological signal according to claim 2, wherein the distributed data server has an analysis unit and a memory unit having the threshold limit value, and in the step D), the analysis unit is used for receiving the processed physiological signals from the pre-processing unit, analyzing the variation of each physiological signal and counting the variations of the physiological signals according to the threshold limit value of the memory unit to generate the statistical values.

4. The method for monitoring a physiological signal according to claim 3, wherein the distributed data server has a calculating unit, and the memory unit has the formula and the sleeping quality information, and wherein the calculating unit is used for receiving the statistical values from the analysis unit, calculating the statistical values via the formula to generate the weighted values, and comparing the weighted values and the sleeping state index to determine the sleeping quality information.

5. The method for monitoring a physiological signal according to claim 4, wherein in the step A), the sensor is used for sensing the bodily movement of the person under sleep test, and in the step D), each statistical values (Bᵢ) about the bodily movement is obtained from counting the time of the variations, which over the threshold limit value, by the analysis unit.

6. The method for monitoring a physiological signal according to claim 5, wherein in the step E), the formula of the memory unit, used for obtaining the weighted statistical values (wBᵢ) about the bodily movement, is as follows:
$${wB}_{i} = ( k - n + 1 ) {\sum }_{n = 1}^{k} {B}_{i - n + 1} / {\sum }_{n = 1}^{k} n$$
, and wherein ‘k’ means the total time of the sleep test, ‘Bᵢ’ means the time of the variations, which over the threshold limit value, in the ‘i th’ minute counting from the start of the sleep test.

7. The method for monitoring a physiological signal according to claim 6, wherein in the step A), the sensor is further used for sensing the leg movement of the person under sleep test, and in the step D), each statistical values (Lᵢ) about the leg movement is obtained from counting the time of the variations, which over the threshold limit value, by the analysis unit.

8. The method for monitoring a physiological signal according to claim 7, wherein in the step E), the formula of the memory unit, used for obtaining the weighted statistical values (wBᵢ) about the bodily movement, is as follows:
$${wL}_{i} = ( k - n + 1 ) {\sum }_{n = 1}^{k} {L}_{i - n + 1} / {\sum }_{n = 1}^{k} n$$
, and wherein ‘k’ means the total time of the sleep test, ‘Lᵢ’ means the time of the variations, which over the threshold limit value, in the ‘i th’ minute counting from the start of the sleep test.

9. The method for monitoring a physiological signal according to claim 8, wherein after the step E), the weighted values about the bodily movement and the leg movement are calculated with a sleep-depth formula of the memory unit by the calculating unit to get a plurality of SleepDepth values, and in the step F), the SleepDepth values generated from the weighted values compares to the sleeping state index to determine the sleeping quality information by the calculating unit.

10. The method for monitoring a physiological signal according to claim 9, wherein the sleep-depth formula is as follows:
SleepDepth value=0.659−0.028wBᵢ−0.026wLᵢ
, and wherein wBᵢ means the weighted values about the bodily movement, wLᵢ means the weighted values about the leg movement.

11. The method for monitoring a physiological signal according to claim 4, wherein in the step A), the sensor is used for receiving the snore of the person under sleep test, and in the step D), each statistical values (Sᵢ) about the snore is obtained from counting the number of the variations, which over the threshold limit value, by the analysis unit.

12. The method for monitoring a physiological signal according to claim 11, wherein the calculating unit is used for counting the numbers of an intermittent snoring of the statistical values to obtain a plurality of intermittent snoring ratios (ISᵢ), wherein the interval time between two snores over 10 seconds is defined the intermittent snoring.

13. The method for monitoring a physiological signal according to claim 12, wherein in the step E), the intermittent snoring ratios (ISᵢ) obtained from the statistical values via the formula to generate a plurality of weighted values by the calculating unit.

14. The method for monitoring a physiological signal according to claim 13, wherein in the step E), the formula of the memory unit, used for obtaining the weighted statistical values (wISᵢ) about the snore, is as follows:
$${wIS}_{i} = ( k - n + 1 ) {\sum }_{n = 1}^{k} {IS}_{i - n + 1} / {\sum }_{n = 1}^{k} n$$
, and wherein ‘k’ means the total time of the sleep test, ‘ISᵢ’ means the number of the variations of the intermittent snoring ratios, which over the threshold limit value, in the ‘i th’ minute counting from the start of the sleep test.

15. The method for monitoring a physiological signal according to claim 14, wherein a voice recorder is electrically connected to the signal processing module, and the voice recorder is used for recording the snore of the person under sleep test.

16. The method for monitoring a physiological signal according to claim 1, wherein in the step H), the sleeping quality information stored in the storage module is read by the electronic device directly.

17. The method for monitoring a physiological signal according to claim 1, wherein the distributed data server has a transmitting module electrically connected to the storage module, and in the step H), the sleeping quality information stored in the storage module is read by the electronic device via internet connected to the transmitting module.

18. The method for monitoring a physiological signal according to claim 17, wherein the electronic device is electrically connected to the transmitting module via an application server.

19. The method for monitoring a physiological signal according to claim 1, wherein after the step G), the sleeping quality information is made into a chart by the distributed data server for reading easily, and the chart is sent to the electronic device regularly.

20. The method for monitoring a physiological signal according to claim 1, wherein after the step G), the sleeping quality information is made into a chart by the distributed data server for reading easily.
